(19) United States
(12) Patent Application Publication
(10) Pub. No.: US 2023/0172735 A1
(43) Pub. Date: Jun. 8, 2023

(54) EXPANDABLE STENT WITH COLLAPSE INHIBITING FEATURES

(71) Applicant: KONINKLIJKE PHILIPS N.V., EINDHOVEN (NL)

(72) Inventor: Adam TSCHIDA, GREENFIELD, MN (US)

(21) Appl. No.: 17/911,679

(22) PCT Filed: Mar. 10, 2021
(86) PCT No.: PCT/EP2021/055961

§ 371 Date: Sep. 15, 2022

Related U.S. Application Data
(60) Provisional application No. 62/990,495, filed on Mar. 17, 2020.

(51) Int. Cl. A61F 2/93

(52) U.S. Cl. CPC A61F 2/93

(57) ABSTRACT

An expandable stent is configured to be deployed in a body lumen. The expandable stent includes a wall, and the wall includes a first longitudinal edge extends and a second longitudinal edge. A ratchet finger is coupled to the wall. The ratchet finger is configured to permit the first longitudinal edge to slide thereover in a first direction upon radial expansion of the expandable stent. The ratchet finger is also configured to engage the first longitudinal edge and inhibit relative motion between the ratchet finger and the first longitudinal edge in a second direction opposite the first direction, and the ratchet finger thereby inhibits radial contraction of the expandable stent.

### CROSS-REFERENCE TO RELATED APPLICATION

None.

### FIELD OF THE DISCLOSURE

The devices and methods described herein generally relate to expandable stents for maintaining patency of body lumens, such as blood vessels.

### BACKGROUND

Expandable stents are commonly used to treat various types of body lumen narrowing conditions, such as deep vein thrombosis, venous compression, atherosclerosis, and the like. Such stents also inhibit restenosis. Two types of stents are commonly used: balloon-expanded stents and self-expanding stents. Balloon-expanded stents are typically formed of stainless steel and are initially carried by a balloon catheter. These stents are positioned in a lumen via the balloon catheter and expand and deploy in the lumen upon inflation of the balloon. However, balloon-expanded stents have various drawbacks, including a relatively low fatigue life and susceptibility to damage when traversing the vasculature of a patient or subject.

In an attempt to address the drawbacks of balloon-expanded stents, self-expanding stents were developed. Such stents are typically formed of a shape memory alloy and normally occupy, or are biased toward, an expanded configuration. Self-expanding stents are constrained in a retracted configuration (for example, by being carried within a sheath) when traversing the vasculature of a subject. When properly positioned in the vasculature, the sheath is withdrawn to permit self-expansion of the stent. More specifically, the stent exerts at radial force at expansion, referred to as chronic outward force. The stent also relies on this force to remain in an expanded configuration, and the stent exerts the force against the vasculature to maintain its position in the vasculature. However, the chronic outward force can lead to stent growth after deployment and radial stent migration into the wall of the vessel. The chronic outward force can also cause vessel trauma, vessel spasm, and a high occurrence of restenosis. Further, relying on this force causes various drawbacks and provides self-expanding stents with competing performance characteristics. For example, increased chronic outward force provides decreased flexibility for self-expanding stents.

Accordingly, improved stents are needed.

### SUMMARY

The present disclosure presents an expandable stent configured to be deployed in a body lumen. The expandable stent includes a wall, and the wall includes an inner surface defining an inner lumen. An outer surface is disposed opposite the inner surface. A first base edge defines a first open end, and the first open end is coupled to the inner lumen. A second base edge defines a second open end, and the second open end is coupled to the inner lumen opposite the first open end. A first longitudinal edge extends from the first open end to the second open end, and a second longitudinal edge extends from the first open end to the second open end. A ratchet finger is coupled to the wall. The ratchet finger is configured to permit the first longitudinal edge to slide thereover in a first direction upon radial expansion of the expandable stent. The ratchet finger is also configured to engage the first longitudinal edge and inhibit relative motion between the ratchet finger and the first longitudinal edge in a second direction opposite the first direction, and the ratchet finger thereby inhibits radial contraction of the expandable stent.

The expandable stent according to the previous paragraph, wherein the ratchet finger extends outwardly from the wall and the first longitudinal edge is disposed outwardly from the inner lumen.

The expandable stent according to any of the previous paragraphs, wherein the expandable stent includes a plurality of ratchet fingers including the ratchet finger, each of the plurality of ratchet fingers being configured to permit the first longitudinal edge to slide thereover in the first direction upon radial expansion of the expandable stent, and each of the plurality of ratchet fingers being configured to engage the first longitudinal edge and inhibit relative motion between the ratchet finger and the first longitudinal edge in the second direction, the plurality of ratchet fingers thereby inhibiting radial contraction of the expandable stent.

The expandable stent according to any of the previous paragraphs, wherein the inner lumen defines a longitudinal axis of the wall, the longitudinal axis extends between the first open end and the second open end, and the plurality of ratchet fingers are offset in a direction parallel to the longitudinal axis.

The expandable stent according to any of the previous paragraphs, wherein the inner lumen further defines a circumferential direction extending about the longitudinal axis, and the plurality of ratchet fingers are offset in the circumferential direction.

The expandable stent according to any of the previous paragraphs, wherein the ratchet finger is cantilevered from the wall.

The expandable stent according to any of the previous paragraphs, wherein the ratchet finger monolithically couples to the wall.

The expandable stent according to any of the previous paragraphs, further including an aperture extending through the wall from the inner surface to the outer surface and disposed adjacent to the ratchet finger.

The present disclosure also presents an expandable stent configured to be deployed in a body lumen. The expandable stent includes a sheet rolled upon itself to define a tube. The tube includes a first open end; a second open end; an inner lumen coupling the first open end to the second open end; a first elongated edge extending from the first open end to the second open end; and a second elongated edge extending from the first open end to the second open end. A ratchet finger is coupled to the tube and is movable relative to the first elongated edge. The ratchet finger is configured to permit the first elongated edge to slide thereover in a first direction upon radial expansion of the stent. The ratchet finger is also configured to engage the first elongated edge and inhibit relative motion between the ratchet finger and the first elongated edge in a second direction opposite the first direction. The ratchet finger thereby inhibits radial contraction of the stent.

The expandable stent according to the previous paragraph, wherein the ratchet finger extends outwardly from the tube and the first longitudinal edge is disposed outwardly from the inner lumen.

The expandable stent according to any of the previous paragraphs, wherein the expandable stent includes a plurality of ratchet fingers including the ratchet finger, each of the plurality of ratchet fingers being configured to permit the first longitudinal edge to slide thereover in the first direction upon radial expansion of the expandable stent, and each of the plurality of ratchet fingers being configured to engage the first longitudinal edge and inhibit relative motion between the ratchet finger and the first longitudinal edge in the second direction, the plurality of ratchet fingers thereby inhibiting radial contraction of the expandable stent.

The expandable stent according to any of the previous paragraphs, wherein the inner lumen defines a longitudinal axis of the tube, the longitudinal axis extends between the first open end and the second open end, and the plurality of ratchet fingers are offset in a direction parallel to the longitudinal axis.

The expandable stent according to any of the previous paragraphs, wherein the inner lumen further defines a circumferential direction extending about the longitudinal axis, and the plurality of ratchet fingers are offset in the circumferential direction.

The expandable stent according to any of the previous paragraphs, wherein the ratchet finger is cantilevered from the tube.

The expandable stent according to any of the previous paragraphs, wherein the ratchet finger monolithically couples to the tube.

The expandable stent according to any of the previous paragraphs, further including an aperture extending through the tube and disposed adjacent to the ratchet finger.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as Xᵢ-Xₙ, Y₁-Yₘ, and Z₁-Zₒ, the phrase is intended to refer to a single element selected from X, Y, and Z, a combination of elements selected from the same class (for example, X₁, and X₂) as well as a combination of elements selected from two or more classes (for example, Y₁ and Zₒ).

The term “a” or “an” entity refers to one or more of that entity. As such, the terms “a” (or “an”), “one or more” and “at least one” may be used interchangeably herein. It is also to be noted that the terms “comprising”, “including”, and “having” may be used interchangeably.

The term “means” as used herein shall be given its broadest possible interpretation in accordance with 35 U.S.C. Section 112(f). Accordingly, a claim incorporating the term “means” shall cover all structures, materials, or acts set forth herein, and all of the equivalents thereof. Further, the structures, materials or acts and the equivalents thereof shall include all those described in the summary, brief description of the drawings, detailed description, abstract, and claims themselves.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure may be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a perspective view of an expandable stent system according to an embodiment of the present disclosure.

FIG. 2 is a perspective view of an expandable stent of the expandable stent system of FIG. 1 in an unexpanded or collapsed configuration.

FIG. 3 is a perspective view of the expandable stent of FIG. 2 in an expanded configuration.

FIG. 4A is a partial cross-sectional view of the expandable stent of FIG. 2 illustrating a ratchet finger in the unexpanded configuration.

FIG. 4B is a partial cross-sectional view of the expandable stent of FIG. 2 illustrating the expandable stent reconfiguring from the unexpanded configuration to the expanded configuration.

FIG. 4C is a partial cross-sectional view of the expandable stent of FIG. 2 illustrating the ratchet finger inhibiting the expandable stent from reconfiguring from the unexpanded configuration to the expanded configuration.

FIG. 5 is a partial side view of the expandable stent of FIG. 2 in the expanded configuration.

FIG. 6 is a partial side view of an expandable stent according to an embodiment of the present disclosure in an expanded configuration.

FIG. 7 is a partial side view of an expandable stent according to another embodiment of the present disclosure in an expanded configuration.

FIG. 8 illustrates a method of manufacturing an expandable stent according to an embodiment of the present disclosure.

It should be understood that the drawings are not necessarily to scale. In certain instances, details that are not necessary for an understanding of the disclosure or that render other details difficult to perceive may have been omitted. It should be understood, of course, that the disclosure is not necessarily limited to the particular embodiments illustrated herein.

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

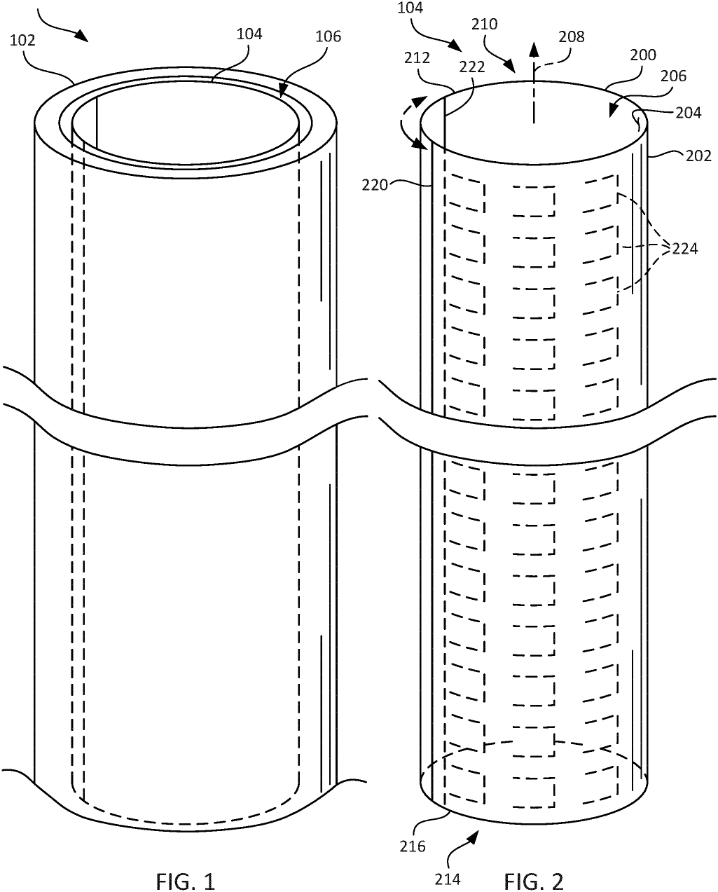

The present disclosure relates generally to devices, systems, and methods for deploying expandable stents to maintain patency of body lumens, such as blood vessels. Referring to FIG. 1, there is shown an exemplary embodiment of an expandable stent system 100. The expandable stent system 100 generally includes a delivery sheath 102 that initially carries an expandable stent 104 in an inner lumen 106. FIG. 1 omits an intermediate section of the expandable stent system 100 for illustrative purposes. Consequently, FIG. 1 does not illustrate the entire length of the expandable stent system 100. The expandable stent 104 may have a length of, for example, about 10 mm - 20 cm. However, the expandable stent 104 may be provided with other lengths depending on the intended application. Further, FIG. 1 omits a proximal section of the delivery sheath 102. At least a portion of such a proximal section remains external to a subject during a stent placement procedure and may be manipulated by a user to guide the expandable stent 104 to a deployment location in the vasculature of the subject, as is well known in the art. The proximal section of the delivery sheath 102 may also be manipulated to deploy the expandable stent 104 from the delivery sheath 102 (that is, translate the expandable stent 104 outwardly relative to the inner lumen 106 of the delivery sheath 102) at the deployment location, as is well known in the art.

Referring now to FIG. 2, the expandable stent 104 is shown immediately upon or after deployment from the delivery sheath 102 (see FIG. 1) and in an unexpanded or collapsed configuration. That is, the expandable stent 104 of FIG. 2 is shown as it is configured within the delivery sheath 102. Generally, the expandable stent 104 includes an open-ended cylindrical wall 200. In some embodiments and as described in further detail below, the expandable stent 104 is a tube that is formed by rolling a sheet upon itself. In the unexpanded configuration, the expandable stent 104 may have a diameter of, for example, about 0.038 inches, 0.052 inches, or 0.065 inches. The wall 200 may comprise one or more of various appropriate materials, such as stainless steel, nitinol, and the like. The wall 200 includes an outer surface 202 and an opposite inner surface 204, and the inner surface 204 defines an inner lumen 206 of the expandable stent 104. The inner lumen 206 defines a longitudinal axis 208 that extends from a first open end 210, defined by a first base edge 212 of the wall 200, to a second open end 214, defined by a second base edge 216 of the wall 200. The inner lumen 206 also defines a circumferential direction 218 that is perpendicular to and extends about the longitudinal axis 208.

To facilitate circumferential and radial expansion of the stent 104, the wall 200 further includes an external, or first, longitudinal edge 220 and an internal, or second, longitudinal edge 222, also referred to as a first elongated edge and a second elongated edge, that both extend between the first open end 210 and the second open end 214. In some embodiments and as described in further detail below, such longitudinal edges 220, 222 may be present due to forming the expandable stent 104 as a sheet rolled upon itself. As shown in FIG. 2, one longitudinal edge is facing one circumferential direction and the other longitudinal edge faces the opposite circumferential direction. For example, the first longitudinal edge 220 is facing a clockwise direction, and the second longitudinal edge 222 is facing a counter-clockwise direction. As the stent expands radially, longitudinal edge 220 moves and rotates in a counter-clockwise direction (while continuing to face the clockwise direction), and longitudinal edge 220 rotates in a clockwise direction (while continuing to face the counter-clockwise direction).

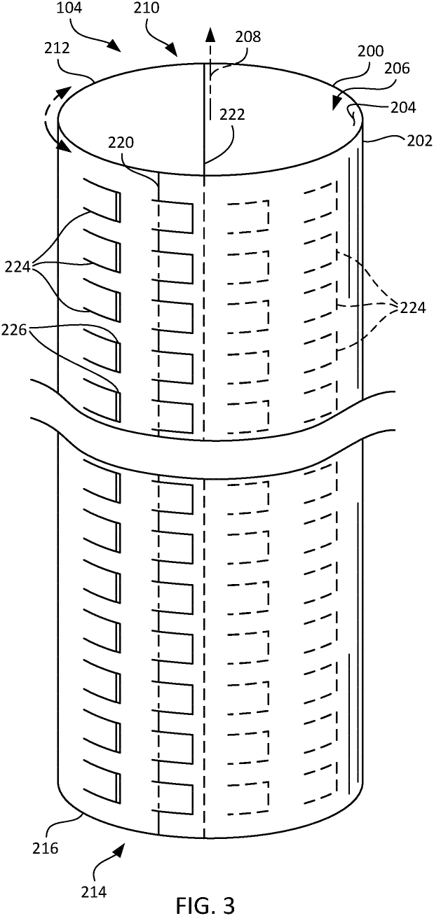

Referring to FIG. 3, the expandable stent 104 is shown after circumferentially and radially expanding to an expanded configuration (or partially expanded configuration). In the expanded configuration the stent 104 has a larger radius and size in the circumferential direction 218 than the unexpanded configuration. In some embodiments, the stent 104 does not expand to a specific size, but instead the stent 104 expands to a size appropriate for engaging the wall of a body lumen and thereby maintaining the position of the stent 104 in the body lumen. The expandable stent 104 may expand to the expanded configuration as adjacent portions of the wall 200 slide, or attempt to unroll, relative to each other. For example, the first longitudinal edge 220 and the second longitudinal edge 222 may slide relative to adjacent portions of the wall 200 to as the stent 104 expands to the expanded configuration. The expandable stent 104 may be expanded to the expanded configuration, for example, via inflation of a balloon catheter (not shown) temporarily positioned with in the inner lumen 206 of the stent 104, or the stent 104 may be self-expanding.

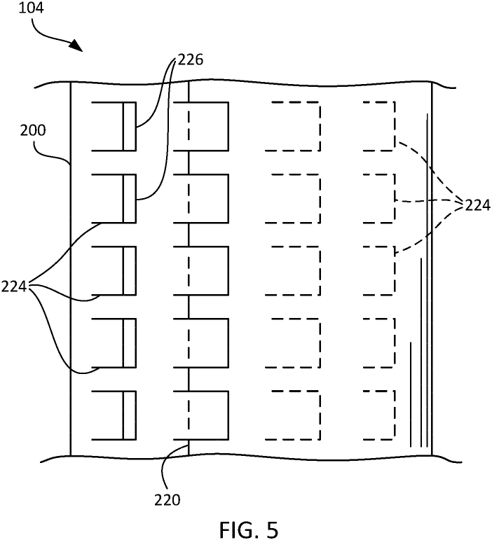

Referring now to FIGS. 2, 3, 4A-4C, and 5, the stent 104 includes one or more collapse inhibiting features, more specifically ratchet fingers 224, that facilitate unidirectional reconfiguration of the stent 104. More specifically, the ratchet fingers 224 permit reconfiguring the stent 104 from the unexpanded configuration to the expanded configuration and inhibit the stent 104 from reconfiguring from the expanded configuration to the unexpanded configuration. As a result, the stent 104 may rely on the ratchet fingers 224 to remain in the expanded configuration and thereby remain in position in the vasculature of a subject and maintain the inner diameter of the vessel. Accordingly, the chronic outward force of the stent 104 may advantageously be reduced or even eliminated.

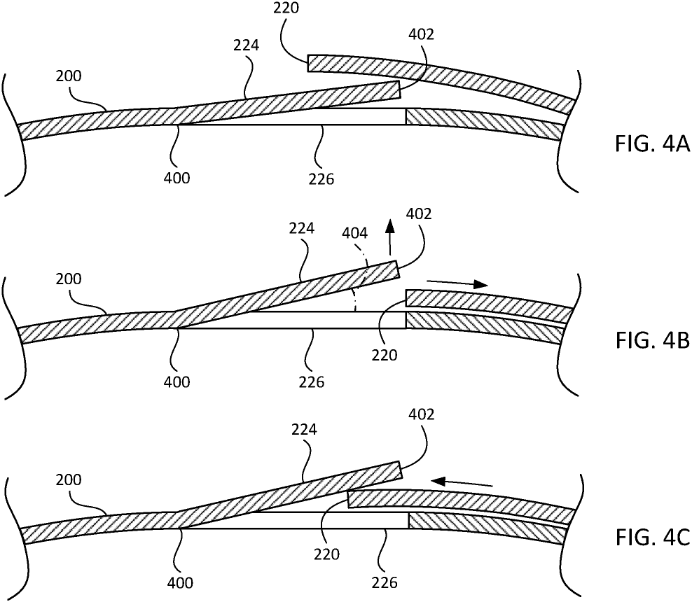

As shown in FIGS. 2 and 4A, the ratchet fingers 224 are covered by adjacent portions of the wall 200 in the unexpanded configuration. As shown in FIG. 4B, the ratchet fingers 224 permit adjacent portions of the wall 200, including the first longitudinal edge 220, to slide thereover in a first direction as the stent 104 is reconfigured from the unexpanded configuration to the expanded configuration. As illustrated, uncovering the ratchet fingers 224 may permit the ratchet fingers 224 to deflect outwardly from the wall 200. As shown in FIGS. 3 and 4C, the ratchet fingers 224 engage the first longitudinal edge 220 and inhibit relative motion between the ratchet finger 224 and the first longitudinal edge 220 in a second direction opposite the first direction. As a result, the ratchet fingers 224 inhibit reconfiguration of the stent 104 from the expanded configuration to the unexpanded configuration.

As illustrated most clearly in FIGS. 4A-4C, the ratchet fingers 224 extend outwardly from the wall 200 of the stent 104. In other embodiments, the ratchet fingers 224 may extend inwardly from the wall 200 of the stent 104 and engage the internal longitudinal edge 222 to inhibit reconfiguration of the stent 104 from the expanded configuration to the unexpanded configuration. In either case, the wall 200 of the stent 104 may include one or more apertures 226, each of the apertures 226 being adjacent to one of the ratchet fingers 224. The apertures 226 and ratchet fingers 224 may both be formed, for example, by laser cutting and bending portions of the wall 200, as described in further detail below.

Referring specifically to FIG. 4B, in some embodiments and as illustrated, each of the ratchet fingers 224 has a first end 400 coupled to the wall 200 and a second, or free, end 402 opposite the first end 400. In some embodiments and as illustrated, the ratchet fingers 224 are monolithically coupled to and cantilevered from the wall 200 at their first ends 400. In some embodiments and as illustrated, the ratchet fingers 224 are normally disposed at acute angles 404 relative to the outer surface 202 of the wall 200 (“normally” and variants thereof referring to situations in which external forces are not applied to component). As a specific example, the acute angles 404 are normally in a range of 10-45 degrees. In some embodiments and as illustrated, the acute angles 404 are disposed in planes perpendicular to the longitudinal axis 208. The acute angles 404 cause the free ends 402 of the ratchets fingers 224 to generally face in a first circumferential direction (for example, the counterclockwise direction, as illustrated). The first longitudinal edge 220 faces a second, opposite circumferential direction (for example, the clockwise direction, as illustrated). As a result, the first longitudinal edge 220 slides over the ratchet fingers 224 and their free end 402 when the first longitudinal edge 220 moves in the first circumferential direction and inhibits sliding in the second circumferential direction.

The ratchet fingers 224 may be provided in various arrangements. In some embodiments, the ratchet fingers 224 are offset in a direction parallel to the longitudinal axis 208. In some embodiments, the ratchet fingers 224 are offset in the circumferential direction 218. In some embodiments and as illustrated, the ratchet fingers 224 are offset in both a direction parallel to the longitudinal axis 208 and the circumferential direction 218.

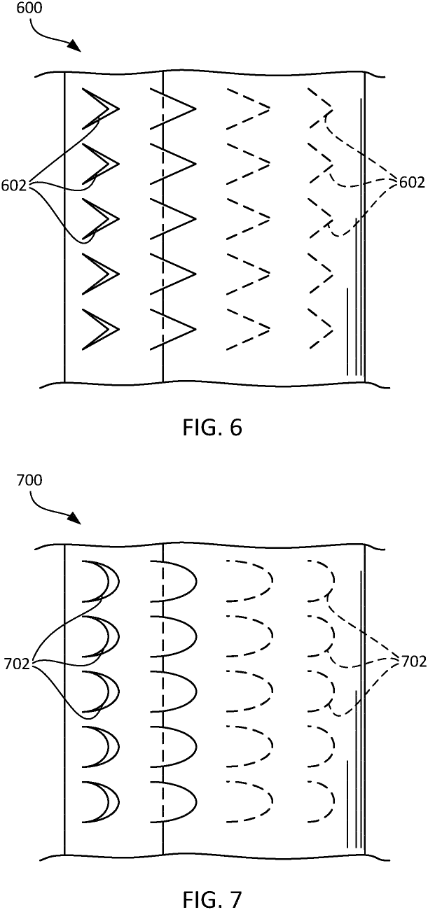

The ratchet fingers 224 may be provided with various shapes. As illustrated in FIG. 2-4C, each ratchet finger 224 may have a rectangular shape. The rectangular shape may have a length (between the first end 400 and the second end 402) of, for example, 0.020 inches to 0.060 inches. The rectangular shape may have a width (perpendicular to the length) of, for example, 0.010 inches to 0.040 inches. In other embodiments, one or more of the ratchet fingers 224 has a different shape. For example and referring to FIG. 6, an exemplary embodiment of expandable stent 600 is illustrated. The expandable stent 600 includes the same or similar features as the expandable stent 104, except that the stent 600 includes one or more ratchet fingers 602 having triangular shapes. As another example and referring to FIG. 7, an exemplary embodiment of expandable stent 700 is illustrated. The expandable stent 700 includes the same or similar features as the expandable stent 104, except that the stent 700 includes one or more ratchet fingers 702 having semi-elliptical shapes.

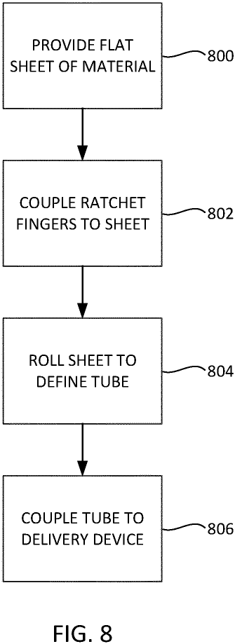

FIG. 8 illustrates an exemplary method of manufacturing an expandable stent, such as any of the stents described herein. The method begins at block 800 by providing a flat sheet of biocompatible material, such as stainless steel, nitinol, and the like. At block 802, one or more ratchet fingers, such as any of the ratchet fingers described herein, are coupled to the sheet. In some embodiments, the ratchet fingers are formed by laser cutting and bending, punching and bending, or welding the fingers to the sheet. At block 804, the sheet is rolled upon itself to define a tube. At block 806, the stent is coupled to a delivery device. More specifically, the stent may be positioned in an inner lumen of a delivery sheath, such as the inner lumen 106 of the delivery sheath 102 described above.

The foregoing discussion has been presented for purposes of illustration and description. The foregoing is not intended to limit the disclosure to the form or forms disclosed herein. In the foregoing Summary for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the description has included description of one or more aspects, embodiments, and/or configurations and certain variations and modifications, other variations, combinations, and modifications are within the scope of the disclosure, for example, as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. An expandable stent configured to be deployed in a body lumen, the expandable stent comprising:
a wall comprising:
an inner surface defining an inner lumen;
an outer surface opposite the inner surface;
a first base edge defining a first open end, the first open end coupled to the inner lumen;
a second base edge defining a second open end, the second open end coupled to the inner lumen opposite the first open end;
a first longitudinal edge extending from the first open end to the second open end; and
a second longitudinal edge extending from the first open end to the second open end; and
a ratchet finger coupled to the wall, the ratchet finger being configured to permit the first longitudinal edge to slide thereover in a first direction upon radial expansion of the expandable stent, and the ratchet finger being configured to engage the first longitudinal edge and inhibit relative motion between the ratchet finger and the first longitudinal edge in a second direction opposite the first direction, the ratchet finger thereby inhibiting radial contraction of the expandable stent.

2. The expandable stent of claim 1, wherein the ratchet finger extends outwardly from the wall and the first longitudinal edge is disposed outwardly from the inner lumen.

3. The expandable stent of claim 1, wherein the expandable stent comprises a plurality of ratchet fingers including the ratchet finger, each of the plurality of ratchet fingers being configured to permit the first longitudinal edge to slide thereover in the first direction upon radial expansion of the expandable stent, and each of the plurality of ratchet fingers being configured to engage the first longitudinal edge and inhibit relative motion between the ratchet finger and the first longitudinal edge in the second direction, the plurality of ratchet fingers thereby inhibiting radial contraction of the expandable stent.

4. The expandable stent of claim 3, wherein the inner lumen defines a longitudinal axis of the wall, the longitudinal axis extending between the first open end and the second open end, and the plurality of ratchet fingers are offset in a direction parallel to the longitudinal axis.

5. The expandable stent of claim 4, wherein the inner lumen further defines a circumferential direction extending about the longitudinal axis, and the plurality of ratchet fingers are offset in the circumferential direction.

6. The expandable stent of claim 3, wherein the inner lumen defines a longitudinal axis of the wall and a circumferential direction extending about the longitudinal axis, and the plurality of ratchet fingers are offset in the circumferential direction.

7. The expandable stent of claim 1, wherein the ratchet finger is cantilevered from the wall.

8. The expandable stent of claim 1, wherein the ratchet finger monolithically couples to the wall.

9. The expandable stent of claim 1, further comprising an aperture extending through the wall from the inner surface to the outer surface and disposed adjacent to the ratchet finger.

10. An expandable stent configured to be deployed in a body lumen, the expandable stent comprising:
a sheet rolled upon itself to define a tube, the tube comprising:
a first open end;
a second open end;
an inner lumen coupling the first open end to the second open end;
a first elongated edge extending from the first open end to the second open end; and
a second elongated edge extending from the first open end to the second open end; and
a ratchet finger coupled to the tube and being movable relative to the first elongated edge, the ratchet finger being configured to permit the first elongated edge to slide thereover in a first direction upon radial expansion of the stent, and the ratchet finger being configured to engage the first elongated edge and inhibit relative motion between the ratchet finger and the first elongated edge in a second direction opposite the first direction, the ratchet finger thereby inhibiting radial contraction of the stent.

11. The expandable stent of claim 10, wherein the ratchet finger extends outwardly from the tube and the first longitudinal edge is disposed outwardly from the inner lumen.

12. The expandable stent of claim 10, wherein the expandable stent comprises a plurality of ratchet fingers including the ratchet finger, each of the plurality of ratchet fingers being configured to permit the first longitudinal edge to slide thereover in the first direction upon radial expansion of the expandable stent, and each of the plurality of ratchet fingers being configured to engage the first longitudinal edge and inhibit relative motion between the ratchet finger and the first longitudinal edge in the second direction, the plurality of ratchet fingers thereby inhibiting radial contraction of the expandable stent.

13. The expandable stent of claim 12, wherein the inner lumen defines a longitudinal axis of the tube, the longitudinal axis extending between the first open end and the second open end, and the plurality of ratchet fingers are offset in a direction parallel to the longitudinal axis.

14. The expandable stent of claim 13, wherein the inner lumen further defines a circumferential direction extending about the longitudinal axis, and the plurality of ratchet fingers are offset in the circumferential direction.

15. The expandable stent of claim 12, wherein the inner lumen defines a longitudinal axis of the tube and a circumferential direction extending about the longitudinal axis, and the plurality of ratchet fingers are offset in the circumferential direction.

16. The expandable stent of claim 10, wherein the ratchet finger is cantilevered from the tube.

17. The expandable stent of claim 10, wherein the ratchet finger monolithically couples to the tube.

18. The expandable stent of claim 10, further comprising an aperture extending through the tube and disposed adjacent to the ratchet finger.
